(19) United States
(12) Patent Application Publication
(10) Pub. No.: US 2017/0343808 A1
(43) Pub. Date: Nov. 30, 2017

(54) CONTROL DEVICE

(71) Applicant: SONY CORPORATION, TOKYO (JP)

(72) Inventors: TETSUYA ASAYAMA, TOKYO (JP); YOSHINORI OOTA, KANAGAWA (JP)

(21) Appl. No.: 15/535,196

(22) PCT Filed: Oct. 20, 2015
(86) PCT No.: PCT/JP2015/079601

§ 371 Date: Jun. 12, 2017

(30) Foreign Application Priority Data
Dec. 26, 2014 (JP) 2014-263957

(51) Int. Cl. G02B 27/01; A61B 5/0478; A61B 5/0408; A61B 5/11; G02C 7/08

(52) U.S. Cl. CPC G02B 27/017; A61B 5/0478; A61B 5/0408; G02B 2027/0178; G02C 7/088; A61B 5/11

(57) ABSTRACT

If the frames of a pair of eyeglasses have a built-in sensor, replacement of the sensor may result in increased effort, cost, and the like. Accordingly, the object is to provide a control device enabling a user to replace the sensor easily. A control device, including: a connecting unit that includes a sensor; and a main unit including a first control unit that conducts processes regarding information obtained from the sensor. The connecting unit is removably connected to each of the main unit and an object mounted on a head of a human body.

### TECHNICAL FIELD

The present disclosure relates to a control device.

### BACKGROUND ART

Recently, technical development related to control devices worn on the head is being conducted. One such control device is a device called a head-mounted display (HMD), for example, and is a device that includes a function of displaying images to a user wearing the HMD. Furthermore, HMDs that acquire sensor information and process the acquired sensor information are also being developed.

For example, Patent Literature 1 discloses an eyeglasses-type operation device in which, when external electronic equipment is connected to eyeglasses-type frames with a built-in sensor, a detection signal obtained by the sensing of the sensor is output to the external electronic equipment.

### CITATION LIST

### Patent Literature

Patent Literature 1: JP 2014-137522A

### DISCLOSURE OF INVENTION

### Technical Problem

However, with the invention disclosed in Patent Literature 1, since the sensor is built into the eyeglasses-type frames, replacing the sensor involves replacing the entire eyeglasses-type frames, and there is a possibility of increased effort, cost, and the like for such replacement. Accordingly, the present disclosure proposes a new and improved control device enabling the user to replace the sensor easily.

### Solution to Problem

According to the present disclosure, there is provided a control device, including: a connecting unit that includes a sensor; and a main unit including a first control unit that conducts processes regarding information obtained from the sensor. The connecting unit is removably connected to each of the main unit and an object mounted on a head of a human body.

### Advantageous Effects of Invention

According to the present disclosure as described above, there is provided a control device enabling the user to replace the sensor easily. Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is a perspective view illustrating an example of an exploded state of a control device according to an embodiment of the present disclosure.

FIG. 2 is a perspective view illustrating an example of a mounted state of a control device according to the present embodiment onto a pair of eyeglasses.

FIG. 3 is a top view illustrating an example of a mounted state of a control device according to the present embodiment onto a pair of eyeglasses.

FIG. 4 is a cross-section view for explaining a configuration of a connecting unit of a control device according to the present embodiment.

FIG. 5 is a perspective view illustrating an example of an exploded state of a control device according to a first modification of the present embodiment.

FIG. 6 is a perspective view illustrating an example of a mounted state of a control device according to a first modification of the present embodiment onto a pair of eyeglasses.

### MODE(S) FOR CARRYING OUT THE INVENTION

Hereinafter, (a) preferred embodiment(s) of the present disclosure will be described in detail with reference to the appended drawings. In this specification and the appended drawings, structural elements that have substantially the same function and structure are denoted with the same reference numerals, and repeated explanation of these structural elements is omitted.

Hereinafter, the description will proceed in the following order.

1. Control device according to embodiment of present disclosure
1-1. Device configuration
1-2. Modifications

2. Conclusion

### <1. Control Device According to Embodiment of Present Disclosure>

A control device 100 according to an embodiment of the present disclosure will be described.

### <1-1. Configuration of Device>

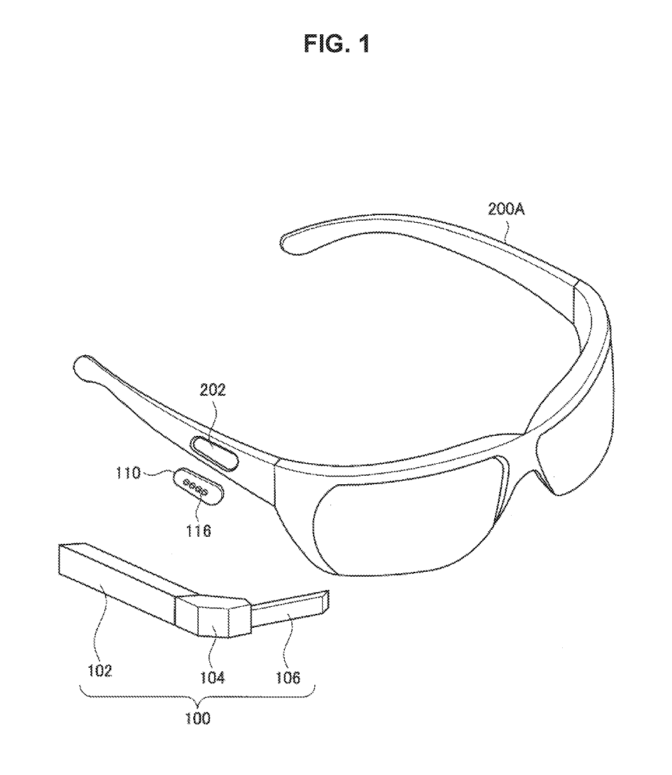

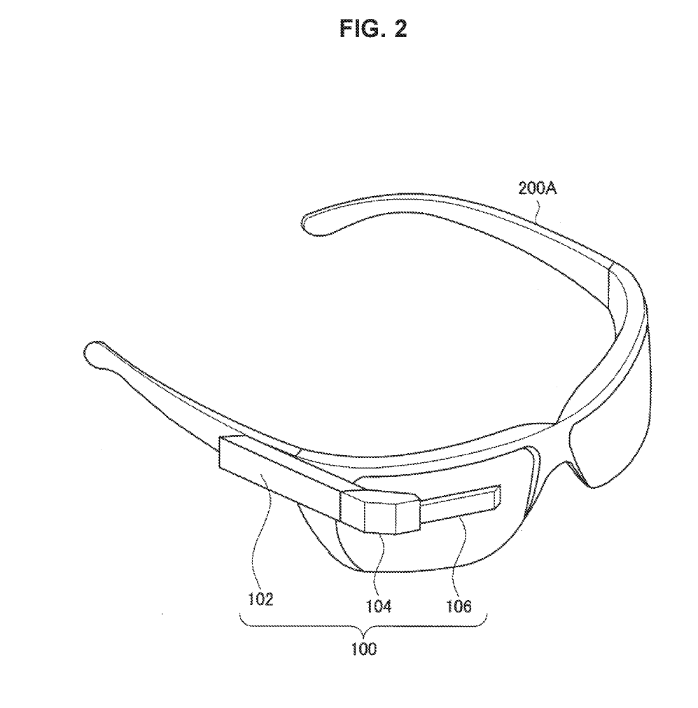

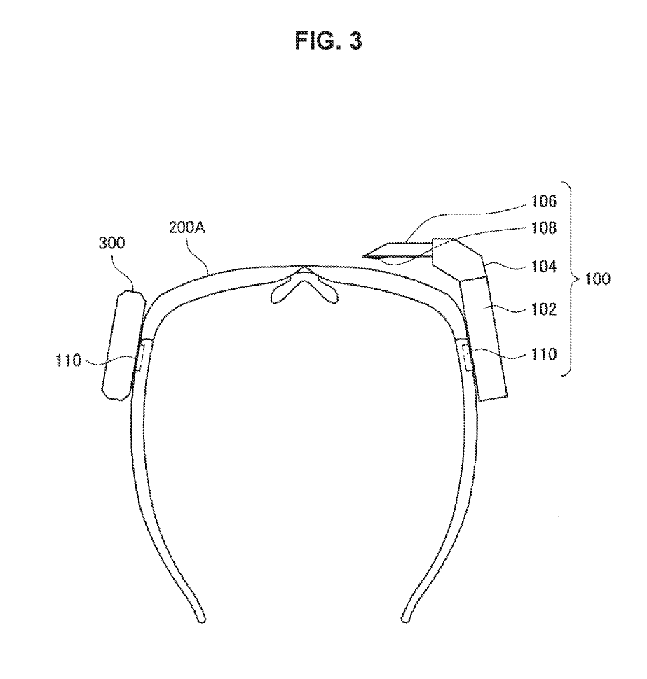

First, a configuration of the control device 100 will be described with reference to FIGS. 1 to 3. FIG. 1 is a perspective view illustrating an example of an exploded state of the control device 100 according to an embodiment of the present disclosure, while FIG. 2 is a perspective view illustrating an example of a mounted state of the control device 100 according to the present embodiment onto a pair of eyeglasses 200A. Also, FIG. 3 is a top view illustrating an example of a mounted state of the control device 100 according to the present embodiment onto the pair of eyeglasses 200A.

As illustrated in FIGS. 1 to 3, the control device 100 is provided with a main unit 102, an arm 104, a lens barrel 106, an eyepiece lens 108, and a connecting unit 110.

The main unit 102 is connected to the arm 104 and the eyeglasses 200A. Specifically, as illustrated in FIG. 1, the lengthwise edge of the main unit 102 is joined to the arm 104, and as illustrated in FIG. 2, one side face of the main unit 102 is joined to the eyeglasses 200A via the connecting unit 110. Note that the eyeglasses 200A may be sports sunglasses or the like, for example.

In addition, the main unit 102 includes a built-in control board for controlling the actions of the control device 100. Specifically, the main unit 102 includes a control board including components such as a central processing unit (CPU) and random access memory (RAM), and is connected to the lens barrel 106 via the arm 104 using signal wires or the like. For example, the control board acts as a first control unit to conduct processes regarding information obtained from a sensor 120 described later (hereinafter also called sensor information). On the control board, processes such as receiving and converting the sensor information, for example, are conducted as the processes. Additionally, the control board also controls the projection of image light in the lens barrel 106, the display of an image according to the image light, and the like.

The arm 104 connects the main unit 102 and the lens barrel 106, and supports the lens barrel 106. Specifically, as illustrated in FIG. 1, the arm 104 is respectively joined to one end of the main unit 102 and one end of the lens barrel 106, and secure the lens barrel 106. Also, the arm 104 includes built-in signal wires for communicating image-related data provided from the main unit 102 to the lens barrel 106.

The lens barrel 106 projects projected image light onto the eyepiece lens. Specifically, the lens barrel 106 includes a projection unit, a light guide unit, and a reflection unit, in which the projection unit projects image light towards the light guide unit, the light guide unit guides the incident image light to the reflection unit, and the reflection unit reflects the arriving image light towards the eyepiece lens. Note that the image according to the projected image light is provided from the main unit 102 via the arm 104.

The eyepiece lens 108 enlarges the image. Specifically, the eyepiece lens is provided at a position where image light is projected from the lens barrel 106, as illustrated in FIG. 1, and refracts image light projected from the lens barrel 106 to magnify the image according to the image light.

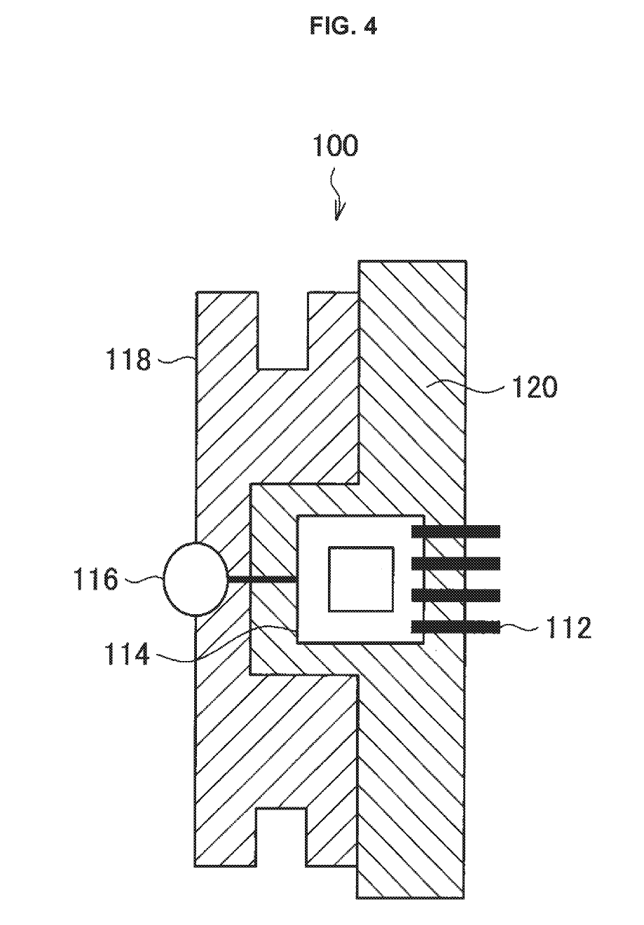

The connecting unit 110 secures the main unit 102 to the eyeglasses 200A. Specifically, the connecting unit 110 includes electrodes 116 as illustrated in FIG. 1, and connects to the main unit 102 via the electrodes 116. Additionally, the eyeglasses 200A include an opening 202 formed to match the shape of the connecting unit 110 as illustrated in FIG. 1, and the connecting unit 110 is fitted into the opening 202. As a result, the main unit 102 and the eyeglasses 200A are connected. The configuration of the connecting unit 110 will be described in further detail with reference to FIG. 4. FIG. 4 is a cross-section view for explaining the configuration of the connecting unit 110 of the control device 100 according to the present embodiment.

The connecting unit 110 is made up of an exterior part 112 and a fitting part 118. As illustrated in FIG. 4, the exterior part 112 includes a control board 114 and the electrodes 116, while the fitting part 118 is made up of the sensor 120.

The exterior part 112 connects directly to the main unit 102. Specifically, the exterior part 112 is joined or fitted with a side face of the main unit 102 via the electrodes 116. For example, the exterior part 112 may be formed from a high-strength material compared to the fitting part 118.

The control board 114 acts as a second control unit to control the actions of the sensor 120. Specifically, the control board 114 controls actions such as the starting and stopping of detection by the sensor 120. Additionally, the control board 114 receives a detection result from the sensor 120, and transmits the detection result to the main unit 102. Specifically, the control board 114 receives a detection signal generated by the sensor 120, and transmits the received detection signal to the main unit 102 via the electrodes 116. The control board 114 may also supply power to the sensor 120.

The electrodes 116 connect the connecting unit 110 to the main unit 102. Specifically, the electrodes 116 electrically connect the control board 114 to the main unit 102, while also structurally connecting the connecting unit 110 to the main unit 102. For example, the electrodes 116 may be made up of two power lines and two signal lines such as Inter-Integrated Circuit (I2C, pronounced I-squared-C), for a total of four lines. Additionally, the main unit 102 is provided with electrodes that mate with the electrodes 116, such as electrodes having concave parts, for example. For this reason, by fitting the convex-shaped electrodes 116 with the concave parts of the electrodes on the main unit 102 side, the control board 114 and the control board in the main unit 102 are electrically connected.

Note that the electrodes 116 may also be made up of five of more electrodes. For example, an interrupt signal line or the like may be provided in addition to the power supply lines and I2C signal lines described above as the electrodes 116. Also, the electrodes 116 may have a concave shape, while the electrodes on the main unit 102 may have convex parts.

The fitting part 118 connects directly to the eyeglasses 200A. Specifically, the fitting part 118 fits into the opening 202 of the eyeglasses 200A, and secures the connecting unit 110 so that the connecting unit 110 becomes a part of the eyeglasses 200A. For example, the fitting part 118 includes a notch in a direction orthogonal to the direction in which the sensor 120 project outward as illustrated in FIG. 4, and this notch fits together with a projecting part of the opening 202 as illustrated in FIG. 2.

In addition, the fitting part 118 includes the sensor 120, and is made from an elastic material. For example, the fitting part 118 may be made from a material such as rubber or plastic. In this case, when the fitting part 118 is mounted closely to the human body, the possibility of making the user feel uncomfortable may be reduced. Furthermore, by having the fitting part 118 be mounted closely to the human body more easily, when the sensor 120 is an optical sensor, external light enters into the sensor 120 less readily, making it possible to minimize drops in the detection accuracy of the sensor 120. Note that since close mounting onto the human body is anticipated, the fitting part 118 may also be made from a sponge-like material.

In addition, the fitting part 118 joins together with the exterior part 112. For example, the fitting part 118 is formed so that the shape of the portion that contacts the exterior part 112 follows the shape of the exterior part 112, and is joined to the exterior part 112.

The sensor 120 detects the surrounding conditions of the connecting unit 110 or the state of the connecting unit 110. Specifically, the sensor 120 is a biosensor that detects biological information about the user wearing the eyeglasses 200A to which the connecting unit 110 is connected. For example, the sensor 120 is an optical sensor or the like that detects pulse, body water, body temperature, body fat, chewing, respiration rate, state of anemia, or oxygen saturation, and outputs a detection result as a signal (hereinafter also called a detection signal). Note that the output detection signal is transmitted to the control board 114 via a signal line. Also, the biosensor may be an ultrasonic sensor or a radio wave sensor.

Furthermore, in the state in which the connecting unit 110 and the eyeglasses 200A are connected, the sensor 120 is provided at a position in close proximity to the human body wearing the eyeglasses 200A. For example, as illustrated in FIG. 4, the sensor 120 is provided on the fitting part 118 so as to project outward from the surface of the fitting part 118 in the opposite direction from the direction in which the electrodes 116 project outward from the exterior part 112.

Note that the sensor 120 may also be provided plurally in the connecting unit 110. For example, two or more sensors 120 may be provided in the fitting part 118, and each of the sensors 120 is connected to the control board 114 by a signal line. In this case, there may be one or multiple control boards 114. For example, the control board 114 may be provided plurally in correspondence with each of the sensors 120, and each of the sensors 120 may be connected to each of the control boards 114 by respective signal lines.

In this way, according to an embodiment of the present disclosure, the control device 100 is provided with a connecting unit 110 that includes a sensor 120, and a main unit 102 including a control board that conducts processes regarding information obtained from the sensor 120. In addition, the connecting unit 110 is removably connected to each of the main unit 102 and the eyeglasses 200A worn on the head of the human body. For this reason, by making the connecting unit 110 that includes the sensor 120 independent from the main unit 102 and the eyeglasses 200A, the sensor 120 may be replaced simply by replacing only the connecting unit 110, making it possible for the user to replace the sensor easily. Additionally, by not having the sensor 120 be built into the eyeglasses 200A, it becomes possible for the user to freely choose the eyeglasses 200A. Additionally, by providing the sensor 120 in the connecting unit 110, it becomes possible to mount the sensor 120 onto the human body by mounting the control device 100, or in other words by simply fitting the connecting unit 110, without attaching the sensor 120 to one's body directly.

In addition, the connecting unit 110 includes a control board 114 that controls the actions of the sensor 120. For this reason, the processing functions related to action control of the sensor 120 in the control board of the main unit 102 become simplified, thereby making it possible to reduce the processing load and power consumption in the control board of the main unit 102.

In addition, the sensor 120 includes a biosensor. For this reason, by mounting the sensor 120 onto the head via the eyeglasses 200A, it becomes possible to acquire biological information about the head.

In addition, in the state in which the connecting unit 110 and the eyeglasses 200A are connected, the sensor 120 is provided at a position in close proximity to the human body wearing the eyeglasses 200A. For this reason, in the case in which the sensor 120 is a biosensor, it becomes possible to improve the detection accuracy of the sensor 120.

In addition, the connecting unit 110 is connected to the eyeglasses 200A as a part of the eyeglasses 200A. For this reason, the control device 100 is connected to the eyeglasses 200A more securely compared to the case in which the control device 100 is connected independently from the eyeglasses 200A, and thus even when the user wearing the control device 100 is exercising, it becomes possible to lower the possibility of the control device 100 becoming removed from the eyeglasses 200A.

In addition, the connecting unit 110 is connected to the main unit 102 via the electrodes 116. For this reason, since a configuration that connects the main unit 102 and the connecting unit 110 does not have to be provided separately, it is possible to reduce the design and manufacturing costs of the control device 100, as well as reduce the complexity of the structure.

### <1-2. Modifications>

The foregoing thus describes an embodiment of the present disclosure. However, the present embodiment is not limited to the foregoing example. Hereinafter, first and modifications of the present embodiment will be described.

### (First Modification)

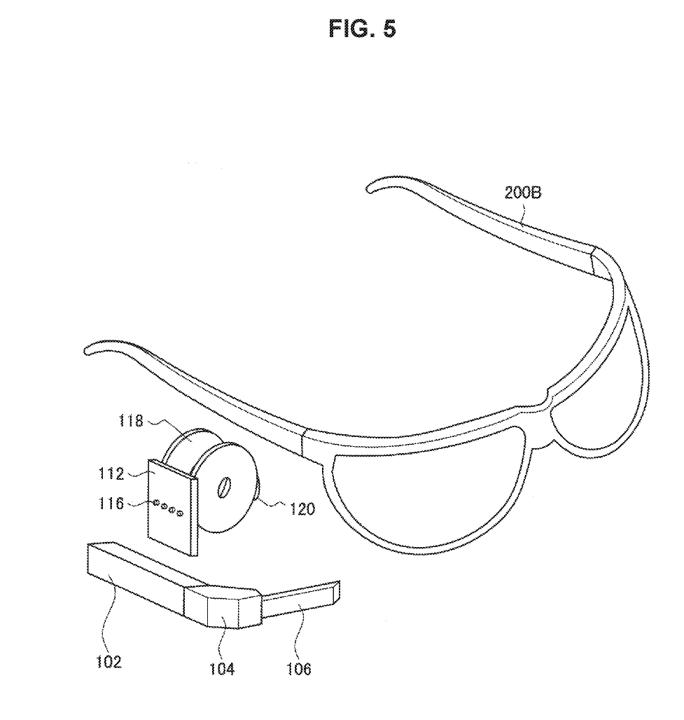

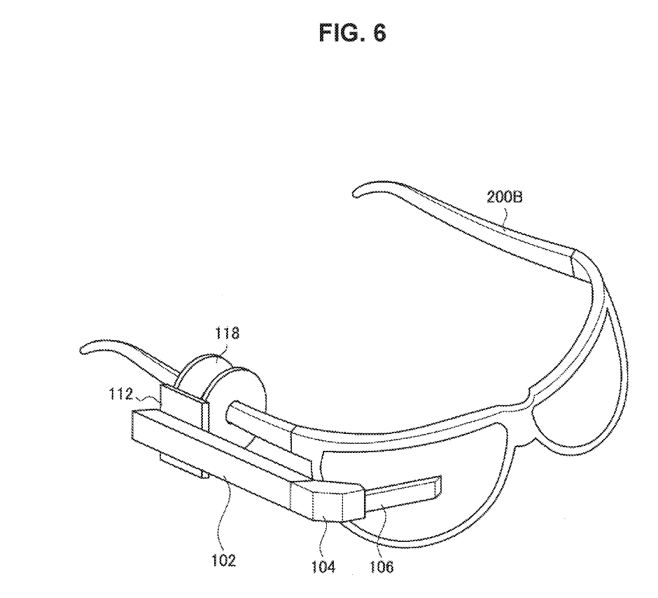

As a first modification of the present embodiment, the control device 100 may also be attachable to a pair of eyeglasses 200 other than the eyeglasses 200A presupposed for the attachment of the control device 100. Specifically, the connecting unit 110 is connected to the eyeglasses 200 in a state independent of the eyeglasses 200. In addition, the control device 100 according to the present modification will be described in detail with reference to FIGS. 5 and 6. FIG. 5 is a perspective view illustrating an example of an exploded state of the control device 100 according to the first modification of the present embodiment, while FIG. 6 is a perspective view illustrating an example of a mounted state of the control device 100 according to the first modification of the present embodiment onto a pair of eyeglasses 200B. Note that the eyeglasses 200B may be typical eyeglasses with lenses.

The fitting part 118 is formed in a ring shape, and joins with the exterior part 112 in a direction orthogonal to the direction going through the hole in the ring. In addition, the fitting part 118 includes the sensor 120 at a position in the opposite direction of the face that joins with the exterior part 112. For example, as illustrated in FIG. 5, the fitting part 118 is formed by two ring-shaped objects lined up in a row with the same direction going through the holes, and joins with the exterior part 112 at the position facing across the holes of the rings from the installation position of the sensor 120. Subsequently, by passing one of the temples of the eyeglasses 200B through each of the holes of the rings, as illustrated in FIG. 6, the control device 100 and the eyeglasses 200B are connected.

Note that although the above describes an example in which there are two rings of the fitting part 118, there may also be one ring, or three or more rings. Also, although the above describes an example in which one of the temples of the eyeglasses 200B is passed through the rings of the fitting part 118, the fitting part 118 may also be made up of an object having a structure that hooks onto one of the temples of the eyeglasses 200B. For example, the fitting part 118 may be made up of an object whereby something is passed vertically through the hole of a ring like the rings illustrated in FIG. 5, or may be made up of a hook-like object.

In this way, according to the first modification of the present embodiment, the connecting unit 110 of the control device 100 is connected to the eyeglasses 200B in a state independent of the eyeglasses 200B. For this reason, by enabling the control device 100 to be attached to a typical pair of eyeglasses 200B lacking a special structure for connecting with the control device 100, it becomes possible for the user to freely choose the eyeglasses 200.

### (Second Modification)

As a second modification of the present embodiment, the control board 114 of the connecting unit 110 may also conduct some of the processes conducted by the control board of the main unit 102. Specifically, the control board 114 conducts the processes regarding the information obtained from the sensor 120. For example, the control board 114 may convert a detection signal in analog format obtained from the sensor 120 into a signal in digital format, or if multiple sensors 120 are provided, the control board 114 may generate another signal on the basis of the detection signal obtained from each of the sensors 120. Furthermore, the control board 114 may generate or alter data on the basis of a detection signal obtained from the sensor 120, and transmit the generated or altered data to the control board of the main unit 102.

In this way, according to the second modification of the present embodiment, the control board 114 of the connecting unit 110 conducts some of the processes conducted by the control board of the main unit 102. For this reason, the processing functions related to the sensor 120 in the control board of the main unit 102 become further simplified, thereby making it possible to further reduce the processing load and power consumption in the control board of the main unit 102.

Note that data generated or altered by the control board 114 may also be deleted after being transmitted to the control board of the main unit 102. In this case, since the data that is generated or altered may correspond to personal information, by deleting such personal information, it becomes possible to ensure the safety of information related to the user who uses the control device 100.

### <2. Conclusion>

Thus, according to an embodiment of the present disclosure, by making the connecting unit 110 that includes the sensor 120 independent from the main unit 102 and the eyeglasses 200A, the sensor 120 may be replaced simply by replacing only the connecting unit 110, making it possible for the user to replace the sensor easily. Additionally, by not having the sensor 120 be built into the eyeglasses 200A, it becomes possible for the user to freely choose the eyeglasses 200A. Additionally, by providing the sensor 120 in the connecting unit 110, it becomes possible to mount the sensor 120 onto the human body by mounting the control device 100, or in other words by simply fitting the connecting unit 110, without attaching the sensor 120 to one's body directly.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

For example, in the foregoing embodiment, the connecting unit 110 is taken to be provided on the control device 100 that displays images, but the present technology is not limited to such an example. For example, the connecting unit 110 may also be provided on a control device 300 that conducts power control. For example, the control device 300 is equipped with a power source, such as a battery, and a power control unit that controls the power source. The control device 300 is connected to the eyeglasses 200 via the connecting unit 110.

Note that both the control device 100 that displays images and the control device 300 that controls power may also be connected to the eyeglasses 200. For example, the control device 100 and the control device 300 may be connected respectively to the frames on the right and left sides of the eyeglasses 200, as illustrated in FIG. 3. In this case, by creating an equilibrium in the weight balance on the left and right frames, it becomes possible to lower the possibility of the eyeglasses 200 becoming removed from the user's head.

In addition, the control device 100 and the control device 300 may be connected by a power line, and power may be supplied from the control device 300 to the control device 100. Furthermore, the control device 100 and the control device 300 may be connected to each other by a signal line and communicate with each other. Additionally, the control device 100 and the control device 300 may also conduct wireless communication.

In addition, although the foregoing embodiment describes an example in which the opening 202 in the eyeglasses 200A penetrates all the way through, the opening 202 of the eyeglasses 200A may also be a depression that does not penetrate all the way through. For example, a wall that covers the opening like a membrane may be provided on the inner side of the frame of the eyeglasses 200A. Note that such a membrane-like wall may be thin enough to allow a measuring wave or the like emitted by the sensor to pass through, for example. In this case, it becomes possible to improve the strength of the frame of the eyeglasses 200A while also enabling the detection by the sensor 120.

Also, the foregoing embodiment describes an example in which the electrodes 116 are arranged in the horizontal direction, but the electrodes 116 may also be arranged in the vertical direction, or may be arranged in both the horizontal direction and the vertical direction.

In addition, multiple electrodes 116 may be arranged as a single group, and electrodes of the main unit 102 that mate with such a group of the electrodes 116 may be provided plurally on the main unit 102. For example, electrodes of the main unit 102 that mate with a group of multiple electrodes 116 arranged in the horizontal direction may be plurality provided in the vertical direction. In this case, since it is sufficient for the group of the electrodes 116 to be connected with any of the electrodes of the main unit 102, the user may select which electrodes of the main unit 102 to connect with the electrodes 116, and thereby be able to adjust the connection position between the main unit 102 and the connecting unit 110.

In addition, the foregoing embodiment describes an example in which the sensor 120 is a biosensor, but the sensor 120 may also be another sensor different from a biosensor. For example, the sensor 120 may also be a sensor that detects information related to the motion or the attitude of the user, such as a sound sensor like a temple microphone, an acceleration sensor, an angular velocity sensor, or a geomagnetic sensor. In addition, intra-body communication electrodes may be provided instead of a biosensor.

In addition, the foregoing embodiment describes an example in which the display method is a method of projecting towards the user's eye, but specifically, the display method may be a pupil division see-through method in which the projected width of the image light is smaller than the pupil diameter, a prism method, a hologram method, or some other display method. Also, the display method may also be another method, such as projecting image light onto a display face provided separately from the control device 100.

In addition, the foregoing embodiment describes an example in which the control device 100 is connected to the eyeglasses 200, but the control device 100 may also be connected to another object mounted to the head. For example, the control device 100 may also be mounted to a hat, a headband, or the like.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.
(1)

A control device, including:

a connecting unit that includes a sensor; and

a main unit including a first control unit that conducts processes regarding information obtained from the sensor, in which

the connecting unit is removably connected to each of the main unit and an object mounted on a head of a human body.
(2)

The control device according to (1), in which

the connecting unit includes a second control unit that controls an action of the sensor.
(3)

The control device according to (2), in which

the second control unit conducts some of the processes conducted by the first control unit.
(4)

The control device according to any one of (1) to (3), in which

the sensor includes a biosensor.
(5)

The control device according to (4), in which

in a state in which the connecting unit and the object are connected, the sensor is provided at a position in close proximity to the human body wearing the object.
(6)

The control device according to any one of (1) to (5) in which

the connecting unit is connected to the object as a part of the object.
(7)

The control device according to any one of (1) to (5), in which

the connecting unit is connected to the object in a state independent from the object.
(8)

The control device according to any one of (1) to (7), in which

the connecting unit is connected to the main unit via an electrode.

### REFERENCE SIGNS LIST

100 control device
102 main unit
104 arm
106 lens barrel
108 eyepiece lens
110 connecting unit
112 exterior part
114 control board
116 electrodes
118 fitting part
120 sensor

1. A control device, comprising:
a connecting unit that includes a sensor; and
a main unit including a first control unit that conducts processes regarding information obtained from the sensor, wherein
the connecting unit is removably connected to each of the main unit and an object mounted on a head of a human body.

2. The control device according to claim 1, wherein
the connecting unit includes a second control unit that controls an action of the sensor.

3. The control device according to claim 2, wherein
the second control unit conducts some of the processes conducted by the first control unit.

4. The control device according to claim 1, wherein
the sensor includes a biosensor.

5. The control device according to claim 4, wherein
in a state in which the connecting unit and the object are connected, the sensor is provided at a position in close proximity to the human body wearing the object.

6. The control device according to claim 1, wherein
the connecting unit is connected to the object as a part of the object.

7. The control device according to claim 1, wherein
the connecting unit is connected to the object in a state independent from the object.

8. The control device according to claim 1, wherein
the connecting unit is connected to the main unit via an electrode.
